(19) United States
(12) Patent Application Publication
(10) Pub. No.: US 2007/0218020 A1
(43) Pub. Date: Sep. 20, 2007

(54) Composition and method for providing protection from dermatitis, from urushiol and/or from sunlight

(76) Inventor: Nestor Sebastian Tomasi, Joliet, IL (US)

Correspondence Address: PATENTS+TMS;A Professional Corporation, 2849 W. Armitage Ave., Chicago, IL, 60647, US

(21) Appl. No.: 11/377,737

(22) Filed: Mar. 16, 2006

(51) Int. Cl. A61K 8/29; A61K 8/27

(52) U.S. Cl. 424/59

(57) ABSTRACT

A formula and a method provide protection from dermatitis, from urushiol and/or from sunlight. The formula and/or a composition has an ultra-violet reflective ingredient, a preservative and/or a moisturizer to protect skin of a user from the dermatitis, the sunlight and/or urushiol. The formula and/or the composition is applied to an area of the skin of the user to form and/or to create a protective layer on the area of the skin of the user. The UV reflective ingredient in the protective layer reflects and/or blocks UV-A rays and/or UV-B rays from the protective layer and/or from the area of the skin of the user. The protective layer is water-resistant to prevent separation and/or to prevent removal of the protective layer from the area of the skin of the user. The protective layer prevents and/or blocks the dermatitis and/or the urushiol from bonding to, from contacting and/or from being absorbed by the area of the skin of the user. The formula protects and/or blocks the skin of the user from effects of the dermatitis, the urushiol and/or the sunlight via the protective layer and/or the UV reflective ingredient.

### BACKGROUND OF THE INVENTION

The present invention generally relates to a composition and a method for providing protection from dermatitis, from urushiol and/or from sunlight. More specifically, the present invention relates to a composition and a method for providing protection from irritants on and/or degradation of an area of skin of a user. The area of the skin of the user may be affected with, for example, the dermatitis, the urushiol and/or the sunlight. The composition may have one or more ultraviolet light (hereinafter “UV”) reflective ingredients to protect and/or to block the area of the skin of the user from exposure to the sunlight. The composition may be applied to the area of the skin of the user to form and/or to create a protective layer on the skin prior to an exposure to the dermatitis, to the urushiol and/or to the sunlight. The protective layer may reflect UV-A rays from and/or UV-B rays from the skin of the user. The protective layer of the composition may prevent and/or may block the chemical irritants, the microbiological irritants, the dermatitis and/or the urushiol from being absorbed by, from contacting, from bonding to and/or from affecting the area of the skin of the user. As a result, the protective layer of the composition may prevent and/or may block the dermatitis, the urushiol and/or the sunlight from affecting the area of skin of the user.

It is generally known that skin of a user may be affected by dermatitis, by urushiol and/or by sunlight. The dermatitis and/or the urushiol may be transferred from a plant to an area of a skin of a user. The dermatitis may be caused from, for example, poison ivy, poison oak, poison sumac and/or the like. The plant may have an active toxin therein which may cause the dermatitis to develop on the skin of the user. The active toxin of the plant may be transferred to the area of the skin of the user during contact of the area of the skin with, for example, the plant. The active toxin of the plant may be oil, such as, for example, the urushiol. The urushiol may bond to the area of the skin and/or may permeate tissue under the area of the skin of the user. As a result, the urushiol may affect the area of the skin and/or the tissue under the area of the skin. Moreover, the active toxin on the affected area of the skin, the organic molecule and/or the urushiol may not be soluble and/or may not be removed with a solvent, such as, for example, water.

Traditionally, a urushiol block is applied to the area of the skin of the user prior to exposing the area of skin to the dermatitis. The urushiol block has a clay derivative, such as, for example, bentoquantum which may form a layer on the area of the skin of the user. The layer of the clay derivative surrounds and/or absorbs the urushiol prior to the urushiol affecting, adhering to and/or permeating the area of the skin or the tissue under the area of the skin of the user. However, the urushiol block and/or the clay derivative may not surround and/or may not absorb all of the urushiol exposed to the area of the skin of the user. Further, the layer of the urushiol block may not cover and/or may not protect the area of skin of the user from exposure to the urushiol. The urushiol block and/or the clay derivative may not prevent the area of skin of the user from absorbing and/or from being affected by the urushiol. As a result, the urushiol may permeate, may irritate and/or may affect the area of the skin of the user to form dermatitis on the area of the skin and/or in the tissue under the area of the skin of the user.

It is also generally known that UV light, such as, for example, sunlight may negatively affect the skin of the user. The UV light has UV-A rays and/or UV-B rays which damage nucleic acids, such as, for example, deoxyribonucleic acid (hereinafter “DNA”) and/or ribonucleic acid (hereinafter “RNA”) of the user. Further, the UV light may also break down folic acid in, may cause irregular pigmentation of and/or may cause leathering of the skin of the user. Still further, the UV light may suppress an immune function of and/or may damage tissue of the skin of the user resulting in wrinkling of, premature aging of and/or cataract formation in the skin of the user. Moreover, the UV light may damage the skin of the user by, for example, tanning, burning and/or skin cancer, such as, for example, squamous cell carcinoma, basal cell carcinoma and/or malignant melanoma.

Traditionally, a sunscreen has a physical reflective ingredient and a chemical absorbing ingredient to protect the skin of the user from the UV-A rays and/or from the UV-B rays. The physical reflective ingredient is an inorganic material for reflecting the UV-A rays and/or the UV-B rays from the skin of the user. The chemical absorbing ingredient is an organic material containing a chemical, such as, for example, benzophenone or oxybenzone to prevent sunburning of the skin of the user by absorbing UV-B rays. The sunscreen may be applied to the area of the skin to form a transparent layer on the area of the skin for protecting the area of the skin of the user from the UV-A rays and/or from the UV-B rays. Moreover, the sunscreen may include a dilutent, an emulsifier, a moisturizer, a fragrance, a colorant, an oil, a preservative and/or the like.

However, the chemical absorbing ingredient may be activated by the UV light to generate, to form and/or to create a free-radical generator, such as, for example, a carcinogen and/or the like. The free-radical generator may bond to a hydrogen atom on the surface of the skin of the user. As a result, the free-radical generator of the chemical absorbing ingredient may form, may cause and/or may result in formation of a skin cancer, such as, for example, melanoma on the surface of the skin of the user.

Further, the preservative of the sunscreen may be, for example, a paraben-based preservative, a formaldehyde-based preservative and/or the like. The preservative may have a carcinogenicity which may cause irritation of the skin of the user and/or may cause a cancer in the tissue of and/or the skin of the user.

The moisturizer and/or the emollient of the sunscreen may be, for example, a petrolatum which may have impurities and/or the like. The impurities of the moisturizer may have a carcinogenicity which may cause cancer in the tissue and/or the skin of the user. Further, the impurities of the moisturizer may cause and/or may result in production of a toxicant in the tissue and/or the skin of the user.

Still further, the moisturizer may be a chemical plasticizer, such as, for example, a phthalate and/or a solvent of the fragrance of the sunscreen. The chemical plasticizer may cause and/or may result in a birth defect in the offspring of the user, an allergic reaction in the user, in a reproductive impairment of the user and/or in liver damage in the user. Moreover, the colorant of the sunscreen may be a carcinogenic which may cause and/or may result in formation of a cancer in the tissue and/or the skin of the user.

A need, therefore, exists for a composition and a method for providing protection from dermatitis, from urushiol and/or from sunlight. Additionally, a need exists for a composition and a method for providing protection from dermatitis, from urushiol and/or from sunlight which may be applied to form and/or to create a protective layer on an area of the skin of the user prior to the area being affected by the dermatitis, the urushiol and/or the sunlight. Further, a need exists for a composition and a method for providing protection from dermatitis, from urushiol and/or from sunlight which may have one or more reflective ingredients and/or one or more inert minerals to reflect and/or to block UV-A rays and/or UV-B rays from the area of the skin of the user. Still further, a need exists for a composition and a method for providing protection from dermatitis, from urushiol and/or from sunlight which may provide a protective layer on skin of the user to prevent and/or to block absorption of urushiol by the skin of the user. Moreover, a need exists for a composition and a method for providing protection from dermatitis, from urushiol and/or from sunlight which may be applied to the skin of the user to moisturize and/or to protect the skin of the user. Furthermore, a need exists for a composition and a method for providing protection from dermatitis, from urushiol and/or from sunlight which may not have a carcinogenic to prevent formation of a cancer in tissue and/or the skin of the user.

### SUMMARY OF THE INVENTION

The present invention generally relates to a composition and a method for providing protection from dermatitis, from urushiol and/or from sunlight. The composition may be applied to an area of skin of a user to form a protective layer for preventing the dermatitis, the urushiol and/or the sunlight from affecting the area of the skin. The composition may have a UV reflective ingredient to reflect and/or to block the sunlight, such as, for example, UV-A rays and/or UV-B rays from the area of the skin of the user and/or to hypo-allergenically affect the skin of the user. Further, the protective layer may prevent and/or may block absorption of urushiol by the skin of the user. As a result, the protective layer of the composition may prevent and/or may block the dermatitis, the urushiol and/or the sunlight from affecting and/or from damaging the area of the skin of the user.

To this end, in an embodiment of the present invention, a composition for providing protection of an area of skin of a user from dermatitis, from urushiol or from sunlight wherein the composition is applied to the area of the skin of the user is provided. The composition has an ultraviolet light reflective ingredient wherein the ultraviolet light reflective ingredient prevents the area of the skin from being affected by the dermatitis, the urushiol and the sunlight wherein the ultraviolet light reflective ingredient reflects the sunlight from the area of the skin of the user wherein the ultraviolet light reflecting ingredient is made from an invert mineral. Further, the composition has a moisturizer wherein the moisturizer softens the area of the skin of the user. Moreover, the composition has a preservative wherein the preservative prevents growth of bacteria wherein the ultraviolet light reflective ingredient is mixed with the moisturizer and the preservative.

In an embodiment, the inert mineral is one of zinc oxide, titanium dioxide or talc.

In an embodiment, the composition has a diluent wherein the diluent is mixed with the ultraviolet light reflective ingredient, the moisturizer and the preservative.

In an embodiment, the preservative is a carcinogenic-free preservative.

In an embodiment, the ultraviolet light reflective ingredient covers the area of the skin of the user wherein the ultraviolet light reflective ingredient blocks the dermatitis and the urushiol from contacting the skin of the user.

In an embodiment, the preservative is phenoxyethanol and an emollient base.

In an embodiment, the composition has an emulsifier wherein the emulsifier is mixed with the ultraviolet light reflective ingredient, the moisturizer and the preservative wherein the emulsifier binds the ultraviolet light reflective ingredient to the moisturizer and the preservative.

In an embodiment, the ultraviolet light reflecting ingredient reflects UV-A rays and UV-B rays from the area of the skin of the user.

In another embodiment of the present invention, a composition for providing protection of an area of skin of a user from dermatitis, from urushiol or from sunlight wherein the composition is applied to the area of the skin of the user is provided. The composition has an ultraviolet light reflective ingredient wherein the ultraviolet light reflective ingredient is one of titanium dioxide, zinc oxide or talc wherein the ultraviolet light reflective ingredient reflects the sunlight from the area of the skin of the user. Moreover, the composition has a moisturizer wherein the moisturizer is resistant to water wherein the ultraviolet light reflective ingredient and the moisturizer are carcinogenic-free and further wherein the moisturizer and the ultraviolet light reflective ingredient are mixed prior to application to the area of the skin of the user.

In an embodiment, the ultraviolet light reflective ingredient blocks the dermatitis and the urushiol from contacting the area of the skin of the user.

In an embodiment, the composition has a preservative wherein the preservative is mixed with the ultraviolet light reflective ingredient and the moisturizer wherein the preservative prevents growth of bacteria.

In an embodiment, the ultraviolet light reflective ingredient reflects UV-A rays and UV-B rays from the area of the skin of the user.

In another embodiment of the present invention, a method for providing protection of an area of skin of a user from dermatitis, from urushiol or from sunlight is provided. The method has the step of formulating a composition wherein the composition has an ultraviolet light reflective ingredient and a preservative wherein the ultraviolet light reflective ingredient and the preservative are carcinogenic-free and further wherein the ultraviolet light reflective ingredient has an inert mineral. Moreover, the method has the step of applying the composition to the area of the skin prior to exposure of the area of the skin to the dermatitis, the urushiol or the sunlight wherein the composition reflects UV-A rays and UV-B rays from the area of the skin and further wherein the composition prevents the dermatitis and the urushiol from contacting the area of the skin.

In an embodiment, the method has the step of adding a diluent wherein the diluent is mixed with the ultraviolet light reflective ingredient and the preservative.

In an embodiment, the method has the step of adding a moisturizer to the composition wherein the moisturizer is resistant to water and further wherein the moisturizer is carcinogenic-free.

In an embodiment, the method has the step of covering the area of the skin of the user with the composition.

In an embodiment, the method has the step of forming a protective layer of the composition on the area of the skin wherein the protective layer prevents the dermatitis, the urushiol and the sunlight from affecting the area of the skin.

In an embodiment, the inert mineral is one of titanium dioxide, zinc oxide or talc.

In an embodiment, the preservative is phenoxyethanol and an emollient base.

In an embodiment, the method has the step of adding an emulsifier to the ultraviolet light reflective agent and the preservative wherein the emulsifier binds the preservative to the ultraviolet light reflective agent.

It is, therefore, an advantage of the present invention to provide a composition and a method for providing protection from dermatitis, from urushiol and/or from sunlight.

Another advantage of the present invention is to provide a composition and a method for providing protection from dermatitis, from urushiol and/or from sunlight which may be applied to an area of skin of a user to form a protective layer for preventing affects from urushiol and/or from sunlight.

And, another advantage of the present invention is to provide a composition and a method for providing protection from dermatitis, from urushiol and/or from sunlight which may moisturize skin of a user and/or may provide protection from UV-A rays and/or UV-B rays to the skin of the user.

Yet another advantage of the present invention is to provide a composition and a method for providing protection from dermatitis, from urushiol and/or from sunlight which may comply with the U.S. Food and Drug Administration skin protection monograph to protect skin of a user from UV-A rays and/or from UV-B rays.

A further advantage of the present invention is to provide a composition and a method for providing protection from dermatitis, from urushiol and/or from sunlight which may prevent premature aging of skin of the user, may prevent tissue damage in the skin of the user and/or may prevent formation of a free radical generator, a carcinogen, a cancer, wrinkles and/or a cataract.

Moreover, an advantage of the present invention is to provide a composition and a method for providing protection from dermatitis, from urushiol and/or from sunlight which may provide a UV reflective ingredient and/or an inert mineral for reflecting UV-A rays and/or UV-B rays from skin of the user.

And, another advantage of the present invention is to provide a composition and a method for providing protection from dermatitis, from urushiol and/or from sunlight which may provide a titanium dioxide, a zinc oxide, a talc and/or the like for reflecting sunlight from skin of the user to prevent formation of a skin cancer.

Yet another advantage of the present invention is to provide a composition and a method for providing protection from dermatitis, from urushiol and/or from sunlight which may provide a protective layer on an area of skin of the user to prevent the urushiol and/or the sunlight from affecting the area of the skin of the user.

Another advantage of the present invention is to provide a composition and a method for providing protection from dermatitis, from urushiol and/or from sunlight which may provide a hypo-allergenic effect to skin of a user.

A further advantage of the present invention is to provide a composition and a method for providing protection from dermatitis, from urushiol and/or from sunlight which may provide a moisturizer to prevent formation of a carcinogen.

Moreover, an advantage of the present invention is to provide a composition and a method for providing protection from dermatitis, from urushiol and/or from sunlight which may prevent allergic reactions.

And, another advantage of the present invention is to provide a composition and a method for providing protection from dermatitis, from urushiol and/or from sunlight which may be transparent, colorless and/or odorless to prevent formation of a cancer.

Yet another advantage of the present invention is to provide a composition and a method for providing protection from dermatitis, from urushiol and/or from sunlight which may prevent reproductive impairments in a user, may prevent liver damage in a user and/or may prevent birth defects in offspring of a user.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention generally relates to a composition and a method for providing protection from dermatitis, from urushiol and/or from sunlight. The composition may have a UV reflective ingredient, a moisturizer, an inert mineral and/or a preservative for preventing and/or for blocking the dermatitis, the urushiol and/or the sunlight from affecting skin of the user. The composition may be applied to an area of the skin of the user to form and/or to create a protective layer on the area of the skin of the user prior to exposing the area to the dermatitis, the urushiol and/or the sunlight.

The area of the skin of the user which may receive the composition and/or the protective layer may be an area of the skin of the body of the user. The composition may be compounded into a form, such as, for example, a cream, an aqueous gel, a spray, a lotion, a lip stick, a powder, a complexion foundation and/or the like. The protective layer which may be applied to the area of the skin of the user may be, for example, transparent, fragrance-free, colorless, odorless and/or the like. It should be understood that the form of the composition may be compounded into any form as known to one of ordinary skill in the art.

The chemical irritants may be derived from an environment and/or from contact with, for example, plants, animals, cosmetics, clothes and/or the like. Further, the chemical irritants may be derived from an acute use, a chronic use or an exposure basis. The chemical irritants may cause a dermatitis, such as, for example, urushiol induced contact dermatitis. The dermatitis may be caused from a plant of the ginkgoaceae family or the anacardiaceae family, such as, for example, poison ivy, poison oak, poison sumac and/or the like. The present invention should not be deemed as limited to the embodiments of a specific dermatitis caused by the chemical irritants and/or a specific plant and/or a specific plant family causing the dermatitis.

The plant may have an active toxin thereon which may cause the dermatitis to develop on the skin of the user. The active toxin of the plant may be, for example, urushiol and/or the like. The protective layer of the composition on the area of the skin of the user may block and/or may prevent the urushiol from contacting, from bonding to and/or from being absorbed by the area of the skin of the user. As a result, the protective layer of the composition may prevent and/or may block the active toxin of the plant from being transferred to, from bonding to, from being absorbed by and/or from affecting the area of the skin of the user during exposure of the active toxin to the area of the skin of the user. The composition may prevent the active toxin and/or the urushiol from bonding to the skin user and/or from permeating tissue under the area of the skin of the user. As a result, the area of the skin and/or the tissue under the area of the skin may be protected from and/or may be blocked from the active toxin and/or the urushiol by the protective layer of the composition which may be applied to the area of the skin of the user.

The composition may have an ultraviolet light (UV) reflective ingredient to prevent and/or to block sunlight and/or a UV light, such as, for example, UV-A rays and/or UV-B rays from damaging and/or from affecting the area of the skin of the user. Further, the UV reflective ingredient may prevent and/or may block long UV-A rays from damaging and/or from affecting the area of the skin of the user. The UV reflective ingredient may be an inorganic material which may contain an inert mineral, such as, for example, zinc oxide, titanium dioxide, talc and/or the like. The UV reflective ingredient may have a weight percentage range of eight percent to fourteen percent of a total composition of the composition. The composition may have a dry condition skin protection factor (hereinafter “SPF”) of, for example, greater than twenty. The composition may have a water resistant SPF of, for example, greater than ten. It should be understood that the UV reflecting ingredient may be any inorganic material having an inert mineral which may reflect the sunlight from the skin of the user.

The composition may have a chelating agent, such as, for example, disodium ethylenediamine tetra-acetic acid (hereinafter “disodium EDTA”) and/or the like. A weight percentage range of 0.05% to 0.15% of the total composition may be the chelating agent. The present invention should not be deemed as limited to the embodiments of a specific chelating agent of the composition.

The composition may have a humectant agent, such as, for example, gylcerin, glyceryl stearate and/or the like. A weight percentage range of four percent to eight percent of the total composition may be the humectant agent. The present invention should not be deemed as limited to the embodiments of a specific humectant agent of the composition. The composition may have a pondus hydrogenii (hereinafter “pH”) range between, for example, six pH and nine pH. The composition may have a viscosity range between sixty-five thousand centipoise per second (hereinafter “CPS”) and eighty-five thousand CPS at twelve revolutions per minute at twenty-five degrees Celsius (hereinafter “C”).

The UV reflective ingredient of the composition may not be absorbed by and/or may not permeate the skin of the user after application to the area of the skin of the user. The UV reflective ingredient of the composition may be refined to a particle size, such as, for example, less than 0.2 microns. The particle size of the UV reflective ingredient may allow the protective layer which may be applied to the area of the skin of the user to be, for example, transparent, colorless, odorless and/or the like. It should be understood that the particle size of the UV reflective ingredient of the composition may be any size as known to one of ordinary skill in the art.

The UV reflective ingredient may reflect the UV light, the UV-A rays and/or the UV-B rays from the skin of the user to protect and/or the block the user from the UV light, the UV-A rays and/or the UV-B rays. The UV reflective ingredient may prevent and/or may block formation of free radical generators and/or carcinogens in the protective layer of the composition which may be applied to the area of the skin of the user. As a result, the UV reflective ingredient may prevent and/or may block development of a cancer of the skin of the user. Further, the UV reflecting ingredient may prevent and/or may block the sunlight from affecting the skin of the user. The composition may have a moisturizer to treat, to moisturize, to condition and/or to soften the skin of the user. The moisturizer may be, for example, shea butter, isopropyl myristate, jojoba oil, glycerin/diethylene glycol/adipate crosspolymer and/or the like. The moisturizer may have a weight percentage range of fifteen percent to twenty-three percent of a total composition of the composition. The composition may increase moisture content of the skin of the user over a duration of time, such as, for example, twenty-four hours. Moreover, the composition may form a film on the skin of the user to provide a barrier of protection against moisture loss of the skin of the user.

The moisturizer may be a petrolatum-free moisturizer and/or a carcinogenic-free moisturizer. Further, the moisturizer may not have impurities which may result in formation of carcinogens and/or toxins in the area of the skin of the user. As a result, the composition and/or the moisturizer may prevent and/or may block development and/or formation of a cancer on the skin of the user or in the tissue of the skin of the user. The moisturizer may adhere to and/or may attract to the skin of the user to resist removal and/or to resist separation of the composition from the skin of the user by, for example, water. As a result, the composition may be water-resistant via the moisturizer of the composition, respectively.

The composition may have a preservative, such as, for example, a natural preservative and/or the like. The preservative may be a paraben-free preservative, a formaldehyde-free preservative and/or the like. The preservative may be, for example, a liquid preservative formulation which may have phenoxyethanol, henoxyethanol and/or an emollient base. The preservative may have a weight percentage range of 0.5 percent to one percent of a total composition of the composition. The preservative may be, for example, a carcinogenic-free preservative to prevent irritation of the skin and/or to prevent and/or to block formation of a cancer on the skin and/or in the tissue of the skin of the user. Further, the preservative may prevent and/or may block growth of, for example, bacteria, yeast and/or the like. The preservative may be prepared in, for example, an aqueous system, an anhydrous system, an emulsion system and/or the like. Moreover, the preservative may enhance the co-solvency of the composition. It should be understood that the preservative may be any paraben-free preservative and/or any formaldehyde-free preservative as known to one of ordinary skill in the art.

The composition may have, for example, a diluent and/or an emulsifier for mixing and/or combining the UV reflective ingredient, the moisturizer and/or the preservative. The diluent may be, for example, deionized water and/or the like. The diluent may have a weight percentage range of fifty percent to sixty percent of a total composition of the composition. The emulsifier may be, for example, xanthan gum, C12-C15 alkyl benzoate, cetearyl alcohol, ceteareth, glycine soja oil, zea mays starch, and/or the like. The emulsifier may have a weight percentage range of three percent to ten percent of a total composition of the composition. The emulsifier may bind and/or may bond the diluent and/or the UV reflective ingredient to the the moisturizer and/or the preservative.

The UV reflective ingredient, the moisturizer and/or the preservative may be combined with, may be added to and/or may be formulated with the diluent and/or the emulsifier to form and/or to create the formula and/or the composition. The composition may be formulated by adding and/or by mixing the UV reflective ingredient, the moisturizer, the preservative, the diluent and/or the emulsifier. A first treatment of the composition may be applied to the area of the skin of the user prior to the area being exposed to the dermatitis, the sunlight and/or the urushiol.

The composition may form and/or may create a protective layer on the skin at the area of the skin of the user. The protective layer may cover and/or may enclose the area of the skin of the user. An unabsorbed portion of the composition may not be absorbed by the skin of the user to form the protective layer. The protective layer which may be formed by the unabsorbed portion of the composition may coat and/or may cover the area of the skin of the user to protect the area of the skin of the user. The unabsorbed portion of the composition may have the UV reflective ingredient therein for reflecting and/or for blocking the UV-A rays and/or the UV-B rays from the protective layer and/or away from the area of the skin of the user. The composition may not cause a sting sensation and/or a burning sensation when applied to the skin of the user. The composition may not irritate and/or may not cause skin sensitivity to the skin of the user when the composition is re-applied to the skin of the user. As a result, the composition may be a non-primary irritant and/or a non-primary sensitizer to the skin of the user.

The protective layer of the composition may prevent and/or may block the dermatitis, the active toxins, the urushiol, the chemical irritants and/or the microbiological irritants from contacting, from being absorbed by and/or from bonding to the area of the skin of the user. Further, the protective layer may reflect and/or may block the UV light, the UV-A rays, the UV-B rays and/or the sunlight from the protective layer and/or the area of the skin of the user which may be cover by the protective layer. The protective layer may be exposed to the solvent, such as, for example, water after being applied to the area of the skin of the user. The protective layer may be resistant to the solvent to prevent separation of the protective layer, the composition from the skin of the user. As a result, the composition may prevent and/or may block the dermatitis, the active toxins, the urushiol, the chemical irritants and/or the microbiological irritants, the UV-A rays, the UV-B rays and/or the sunlight from affecting and/or from damaging the skin of the user.

In an embodiment, compounding of the composition may occur in a first container and/or a second container. The first container and/or the second container may be, for example, a stainless steel tank. A diluent, such as, for example, the deionized water may be added to the first container and/or may be agitated therein. The present invention should not be deemed as limited to the embodiments of a specific container used during the compounding of the composition.

The xanthan gum may be added to the diluent in the first container to form a batch of the composition. The xanthan gum may be mixed with or may dispersed into the diluent in the first container. The batch of the composition inside the first container may be heated to a first temperature range between seventy-five degrees C and eighty degrees C. The glycerin and/or the disodium EDTA may be added to and/or may be mixed into the batch of the composition inside the first container to form a water phase of the composition.

The isopropyl myristate, the jojoba oil, the C12-C15 alkyl benzoate, the glyceryl stearate, the shea butter, cetearyl alcohol, the ceteareth and/or the glycerin/diethylene glycol/adipate crosspolymer may be added to the second container to form an oil phase of the composition. The oil phase may be heated to the first temperature range. The water phase and the oil phase may be uniform and/or may be heated to the first temperature range. The oil phase in the second container may be added to the water phase of the composition in the first container. The oil phase and the water phase inside the first container may be mixed for a time period between, such as, for example, fifteen minutes and twenty minutes. As a result, the batch of the composition inside the first container having the water phase and the oil phase of the composition may be homogeneous.

The batch of the composition inside the first container may be mixed by a mixing device, such as, for example, a sweep mixer and/or the like. The batch of the composition inside the first container may be cooled to a second temperature range between, for example, sixty-five degrees C and seventy degrees C. The zinc oxide and/or the titanium dioxide may be added to and/or may be dispersed into the batch of the composition inside the first container.

The glycine soja oil and/or the zea mays starch may be added to and/or may be mixed with the batch of the composition inside the first container. The batch of the composition may be mixed via the mixing device until the batch may not have lumps in the batch of the composition. The batch of the composition may be cooled to a third temperature range between, for example, forty degrees C and forty-five degrees C. The preservative may be added to and/or may be mixed with the batch of the composition inside the first container. The batch of the composition may be mixed for a duration of time between, for example, ten minutes and fifteen minutes. As a result, the preservative may be dispersed within the batch of the composition. The batch of the composition may be collected from the first container.

The composition may have the UV reflective ingredient, the preservative and/or the moisturizer to protect the skin of the user from the dermatitis, the urushiol and/or the sunlight. The composition may be applied to the area of the skin of the user to form and/or to create a protective layer on the area of the skin of the user. The UV reflective ingredient in the protective layer may reflect and/or may block the UV-A rays and/or the UV-B rays from the protective layer and/or from the area of the skin of the user. The protective layer may be water-resistant to prevent separation and/or to prevent removal of the composition from the area of the skin of the user. The protective layer may prevent and/or may block the dermatitis and/or the urushiol from bonding to, from contacting and/or from being absorbed by the area of the skin of the user. As a result, the composition may protect and/or may block the skin of the user from the effects of the dermatitis, the urushiol and/or the sunlight via the protective layer and/or the UV reflective ingredient of the composition.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

I claim:

1. A composition for providing protection of an area of skin of a user from dermatitis, from urushiol or from sunlight wherein the composition is applied to the area of the skin of the user, the composition comprising:
an ultraviolet light reflective ingredient wherein the ultraviolet light reflective ingredient prevents the area of the skin from being affected by the dermatitis, the urushiol and the sunlight wherein the ultraviolet light reflective ingredient reflects the sunlight from the area of the skin of the user wherein the ultraviolet light reflecting ingredient is made from an invert mineral;
a moisturizer wherein the moisturizer softens the area of the skin of the user; and
a preservative wherein the preservative prevents growth of bacteria wherein the ultraviolet light reflective ingredient is mixed with the moisturizer and the preservative.

2. The composition of claim 1 wherein the inert mineral is one of zinc oxide, titanium dioxide or talc.

3. The composition of claim 1 further comprising:
a diluent wherein the diluent is mixed with the ultraviolet light reflective ingredient, the moisturizer and the preservative.

4. The composition of claim 1 wherein the preservative is a carcinogenic-free preservative.

5. The composition of claim 1 wherein the ultraviolet light reflective ingredient covers the area of the skin of the user wherein the ultraviolet light reflective ingredient blocks the dermatitis and the urushiol from contacting the skin of the user.

6. The composition of claim 1 wherein the preservative is phenoxyethanol and an emollient base.

7. The composition of claim 1 further comprising:
an emulsifier wherein the emulsifier is mixed with the ultraviolet light reflective ingredient, the moisturizer and the preservative wherein the emulsifier binds the ultraviolet light reflective ingredient to the moisturizer and the preservative.

8. The composition of claim 1 wherein the ultraviolet light reflecting ingredient reflects UV-A rays and UV-B rays from the area of the skin of the user.

9. A composition for providing protection of an area of skin of a user from dermatitis, from urushiol or from sunlight wherein the composition is applied to the area of the skin of the user, the composition comprising:
an ultraviolet light reflective ingredient wherein the ultraviolet light reflective ingredient is one of titanium dioxide, zinc oxide or talc wherein the ultraviolet light reflective ingredient reflects the sunlight from the area of the skin of the user; and
a moisturizer wherein the moisturizer is resistant to water wherein the ultraviolet light reflective ingredient and the moisturizer are carcinogenic-free and further wherein the moisturizer and the ultraviolet light reflective ingredient are mixed prior to application to the area of the skin of the user.

10. The composition of claim 9 wherein the ultraviolet light reflective ingredient blocks the dermatitis and the urushiol from contacting the area of the skin of the user.

11. The composition of claim 9 further comprising:
a preservative wherein the preservative is mixed with the ultraviolet light reflective ingredient and the moisturizer wherein the preservative prevents growth of bacteria.

12. The composition of claim 9 wherein the ultraviolet light reflective ingredient reflects UV-A rays and UV-B rays from the area of the skin of the user.

13. A method for providing protection of an area of skin of a user from dermatitis, from urushiol or from sunlight, the method comprising the steps of:
formulating a composition wherein the composition has an ultraviolet light reflective ingredient and a preservative wherein the ultraviolet light reflective ingredient and the preservative are carcinogenic-free and further wherein the ultraviolet light reflective ingredient has an inert mineral; and
applying the composition to the area of the skin prior to exposure of the area of the skin to the dermatitis, the urushiol or the sunlight wherein the composition reflects UV-A rays and UV-B rays from the area of the skin and further wherein the composition prevents the dermatitis and the urushiol from contacting the area of the skin.

14. The method of claim 13 further comprising the step of:
adding a diluent wherein the diluent is mixed with the ultraviolet light reflective ingredient and the preservative.

15. The method of claim 13 further comprising the step of:
adding a moisturizer to the composition wherein the moisturizer is resistant to water and further wherein the moisturizer is carcinogenic-free.

16. The method of claim 13 further comprising the step of:
covering the area of the skin of the user with the composition.

17. The method of claim 13 further comprising the step of:
forming a protective layer of the composition on the area of the skin wherein the protective layer prevents the dermatitis, the urushiol and the sunlight from affecting the area of the skin.

18. The method of claim 13 wherein the inert mineral is one of titanium dioxide, zinc oxide or talc.

19. The method of claim 13 wherein the preservative is phenoxyethanol and an emollient base.

20. The method of claim 13 further comprising the step of:
adding an emulsifier to the ultraviolet light reflective agent and the preservative wherein the emulsifier binds the preservative to the ultraviolet light reflective agent.
